(19) United States
(12) Patent Application Publication
(10) Pub. No.: US 2005/0180936 A1
(43) Pub. Date: Aug. 18, 2005

(54) Keratin fiber make-up composition with long-term stability

(76) Inventor: Karl Pays, Saint-Maurice (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/029,501

(22) Filed: Jan. 6, 2005

Related U.S. Application Data
(60) Provisional application No. 60/537,542, filed on Jan. 21, 2004.

(30) Foreign Application Priority Data
Jan. 6, 2004 (FR) 04 50015

(51) Int. Cl. A61K 7/06; A61K 7/11

(52) U.S. Cl. 424/70.11; 424/70.16; 424/70.17

(57) ABSTRACT

The present disclosure relates to a composition for the care and/or make-up of keratin fibers comprising, in a solvent medium, (i) at least one film-forming polymer, (ii) at least one polymer comprising at least one styrene unit, wherein the at least one polymer is different from the at least one film-forming polymer (i), and (iii) at least one additional polymer soluble in the solvent medium comprising at least one crystallizable part, wherein the at least one additional polymer is different from the at least one film-forming polymer (i). The present disclosure further relates to the method of making-up keratin fibers wherein the composition is applied to the keratin fibers.

This non provisional application claims the benefit of French Application No. 04 50015 filed on Jan. 6, 2004 and U.S. Provisional Application No. 60/537,542 filed on Jan. 21, 2004.

The present disclosure, in one embodiment, relates to the make-up of keratin substances, for example, keratin fibers such as eyelashes, eyebrows and hair, and further relates to the make-up of eyelashes.

The composition according to the disclosure may take the form of a mascara, an eyebrow product, an eyeliner, or a hair make-up product. In one embodiment, the disclosure relates to a mascara. For example, the composition may be a make-up composition, a composition to be applied over or under a make-up, such as those known as a topcoat or basecoat, or an eyelash treatment composition.

In general, compositions for the make-up of keratin fibers, such as eyelashes, comprise at least one wax or a mixture of waxes dispersed in a liquid phase. In some cases, the specific application properties that are sought for the compositions, for example their fluidity, their covering capacity, their curving capacity, their thickening capacity (also referred to as bulking or make-up capacity) and their stability, are mainly adjusted via the amount of wax and other non-volatile ingredients.

One aspect of the present disclosure is to propose a cosmetic composition for the care and/or make-up of keratin fibers which may possess improved properties in terms of stability.

For example, prior art compositions for the make-up of keratin fibers, such as eyelashes, may not afford a film of significant rubbing resistance. These films generally tend to disintegrate, at least in part, for example, by crumbling or smearing. Partial crumbling of the film results in a substantial loss of intensity of the make-up color, and this lost intensity may only be replaced by reapplying the composition to the already made-up substrate. As far as smearing of the film is concerned, this may also result in the formation of a ring in the vicinity of the made-up zone, which for obvious aesthetic reasons may be undesirable.

Unexpectedly, the composition of the present disclosure may effectively overcome at least one of the above-mentioned drawbacks, e.g., this lack of stability with a novel composition for the care and/or make-up of keratin fibers.

In one embodiment, the present disclosure relates to a cosmetic composition for the care and/or make-up of keratin fibers comprising, in a solvent medium, (i) at least one film-forming polymer, (ii) at least one polymer comprising at least one styrene group, wherein the at least one polymer is different from the at least one film-forming polymer (i), and (iii) at least one additional polymer soluble in the solvent medium comprising at least one crystallizable part, wherein the at least one additional polymer is different from the at least one film-forming polymer (i).

The disclosure, thus, provides that the combined use of these elements in a cosmetic formulation, for example, of the mascara type, may make it possible, i.e., to prolong the stability of the corresponding make-up film, e.g., having a stability of at least two days. As is apparent from the Examples below, the water resistance of this film, for example, may be enhanced. Finally, at least one improvement may be obtained in conjunction with a smoother appearance of the applied film, probably as a result of a better dispersion of the particles of waxes and pigments in the formulation according to the disclosure.

The present disclosure further relates to a method of making up keratin fibers wherein a composition as defined above is applied to the keratin fibers, such as eyelashes.

### Solvent Medium

In one embodiment, the present disclosure relates, for example, to the field of keratin fiber make-up compositions with a low content of water and/or water-soluble solvents, known as waterproof mascaras, which take the form of a dispersion of wax(es) in non-aqueous solvents.

Thus, in one embodiment, the composition according to the disclosure comprises a non-aqueous solvent medium.

This solvent medium may be capable of forming a continuous phase and, as its name indicates, comprises at least one solvent, such as a non-aqueous solvent, which is generally at least one volatile, water-insoluble compound that is liquid at room temperature and atmospheric pressure.

As used herein, “volatile compound” is understood as meaning any compound (or non-aqueous medium) that is capable of evaporating in less than one hour, at room temperature and atmospheric pressure, when in contact with the skin or keratin fiber. The volatile compound is a volatile cosmetic compound that is liquid at room temperature and, for example, whose vapour pressure is non-zero at room temperature and atmospheric pressure and ranges, for example, from 0.13 Pa to 40,000 Pa (10⁻³ mmHg to 300 mmHg), such as from 1.3 Pa to 13,000 Pa (0.01 mmHg to 100 mmHg) and, further for example, from 1.3 Pa to 1300 Pa (0.01 mmHg to 10 mmHg).

Conversely, as used herein, “non-volatile compound” is understood as meaning a compound that remains on the skin or keratin fiber for at least several hours at room temperature and atmospheric pressure and, for example, whose vapor pressure is below 10⁻³ mmHg (0.13 Pa).

The content of the at least one volatile, water-insoluble compound that is liquid at room temperature ranges from 5% to 95%, for example, from 5% to 55%, such as from 10% to 80% and, further for example, 30% to 70% by weight, based on the total weight of the composition.

For example, the at least one volatile, water-insoluble compound that is liquid at room temperature may be a cosmetically acceptable oil or organic solvent. As used herein, the expression “cosmetically acceptable” is understood as meaning a compound whose use is compatible with application to keratin fibers and the skin.

Of course, the solvent medium of the composition according to the disclosure may comprise a mixture of such compounds.

The volatile oils may be chosen from hydrocarbon oils, silicone oils, fluorinated oils, and mixtures thereof.

As used herein, “hydrocarbon oil” is understood as meaning an oil that comprises mainly hydrogen and carbon atoms and optionally oxygen, nitrogen, sulphur, and phosphorus atoms. The volatile hydrocarbon oils may be chosen from hydrocarbon oils having from 8 to 16 carbon atoms, for example, branched C₈-C₁₆ alkanes such as C₈-C₁₆ isoalkanes of petroleum origin (also called isoparaffins), like isododecane (also called 2,2,4,4,6-pentamethylheptane), isodecane, isohexadecane and, e.g., the oils sold under the trade names “Isopars®” and “Permetyls®”, branched C₈-C₁₆ esters, isohexyl neopentanoate, and mixtures thereof. It may also be possible to use other volatile hydrocarbon oils such as petroleum distillates, for example, those sold under the name “Shell Solt®” by SHELL.

Other volatile oils which may be used are volatile silicones, for example, volatile linear or cyclic silicone oils, such as those having a viscosity of ≦6 centistokes (6.10⁻⁶ m²/s) and, for example, from 2 to 10 silicon atoms, these silicones optionally comprising alkyl or alkoxy groups having from 1 to 22 carbon atoms. As volatile silicone oils which may be used in the disclosure, there may be mentioned, for example, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

It may also be possible, for example, to use fluorinated volatile organic solvents such as nonafluoromethoxybutane or perfluoromethylcyclopentane.

In one embodiment of the compositions according to the disclosure, the at least one volatile, water-insoluble compound that is liquid at room temperature is chosen from volatile hydrocarbon oils having from 8 to 16 carbon atoms, and mixtures thereof.

The non-aqueous solvent medium may also comprise at least one non-volatile, water-insoluble compound that is liquid at room temperature, such as at least one non-volatile oil, which can be chosen, for example, from non-volatile hydrocarbon, silicone, and fluorinated oils.

The following may be mentioned, for example, as non-volatile hydrocarbon oils chosen from:

hydrocarbon oils of vegetable origin, such as triglycerides comprising fatty acid esters of glycerol wherein the fatty acids may have chain lengths varying from C₄ to C₂₄, it being possible for the latter to be linear and branched and saturated and unsaturated; these oils are, for example, wheatgerm, sunflower, grapeseed, sesame, maize, apricot, castor, shea, avocado, olive, soya, sweet-almond, palm, colza, cottonseed, hazelnut, macadamia, jojoba, alfalfa, poppy, pumpkin, gourd, blackcurrant, evening primrose, millet, barley, quinoa, rye, safflower, candlenut, passiflora and muscat rose oils; and caprylic/capric triglycerides such as those sold by STEARINERIES DUBOIS and those sold under the names “Miglyol 810®”, “812®” and “818®” by DYNAMIT NOBEL;

synthetic ethers having from 10 to 40 carbon atoms;

linear and branched hydrocarbons of mineral and synthetic origin, such as petrolatum, polydecenes, hydrogenated polyisobutene such as parleam, squalane, and mixtures thereof;

synthetic esters such as oils of the formula R₁COOR₂, wherein R₁ is a radical of a linear and branched fatty acid comprising from 1 to 40 carbon atoms and R₂ is a hydrocarbon chain, such as a branched hydrocarbon chain, comprising from 1 to 40 carbon atoms, with the proviso that R₁+R₂≧10, for example, purcellin oil (cetostearyl octanoate), isopropyl myristate, isopropyl palmitate, the benzoate of a C₁₂ to C₁₅ alcohol, hexyl laurate, diisopropyl adipate, isononyl isononanoate, 2-ethylhexyl palmitate, isostearyl isostearate, the octanoates, decanoates and ricinoleates of alcohols and polyalcohols, such as propylene glycol dioctanoate; hydroxylated esters such as isostearyl lactate and diisostearyl malate; and pentaerythritol esters;

fatty alcohols that are liquid at room temperature and comprise a branched and unsaturated carbon chain having from 12 to 26 carbon atoms, such as octyldodecanol, isostearyl alcohol, oleyl alcohol, 2-hexyldecanol, 2-butyloctanol and 2-undecylpentadecanol;

higher fatty acids such as oleic acid, linoleic acid and linolenic acid; and mixtures thereof.

The at least one non-volatile silicone oil which may be used in the composition according to the disclosure may be non-volatile polydimethylsiloxanes (PDMS), polydimethylsiloxanes comprising alkyl or alkoxy groups that are pendent and at the end of the silicone chain and each have from 2 to 24 carbon atoms, and phenylated silicones such as phenyltrimethicones, phenyldimethicones, phenyltrimethylsiloxydiphenyl-siloxanes, diphenyldimethicones, diphenylmethyidiphenyltrisiloxanes, and 2-phenylethyl trimethylsiloxysilicates.

The fluorinated oils which may be used in the composition of the disclosure are, for example, fluorosilicone oils, fluorinated polyethers, and fluorinated silicones such as those described in European Application No. EP-A-847 752.

In one embodiment, the compositions according to the disclosure may comprise water and/or at least one water-soluble solvent.

As used herein, “water-soluble solvent” is understood as meaning a compound that is liquid at room temperature and miscible with water (miscibility with water greater than 50% by weight at 25° C. and atmospheric pressure).

The at least one water-soluble solvent, which may be used in the compositions according to the disclosure, may be generally also volatile.

The following may be mentioned, for example, among the at least one water-soluble solvent, which may be used in the compositions according to the disclosure: lower monoalcohols having from 1 to 5 carbon atoms, such as ethanol and isopropanol, glycols having from 2 to 8 carbon atoms, such as ethylene glycol, propylene glycol, 1,3-butylene glycol and dipropylene glycol, C₃ and C₄ ketones and C₂-C₄ aldehydes.

The water and/or the at least one water-soluble solvent may be introduced as such into the formulation according to the disclosure or may be incorporated therein by way of at least one ingredient that form part of the composition. Thus, water may be introduced into the composition, for example, by way of the introduction of an aqueous dispersion of particles of a polymer that is useful, e.g., as a film-forming agent.

As far as waterproof mascaras are concerned, however, the content of water and/or the at least one water-soluble solvent in the composition may be less than or equal to 20% and is generally greater than or equal to 0.5% by weight, based on the total weight of the composition.

The content of water and/or the at least one water-soluble solvent in the compositions of the disclosure vary, for example, from 1% to 18% and, further for example, from 2 to 15% by weight, based on the total weight of the composition.

### Film-Forming Polymer

The composition according to the disclosure comprises at least one film-forming polymer.

As used herein, “film-forming polymer” is understood as meaning a polymer that is capable of forming a continuous and adhesive film on a substrate, such as on keratin substances, either by itself or in the presence of an auxiliary filmifying agent.

In general, the at least one film-forming polymer may be present in the composition according to the disclosure in a content ranging from 0.1% to 55% by weight, such as from 0.5% to 40% by weight and, further for example, from 1% to 30% by weight, based on the total weight of the composition.

For example, this amount may be liable to vary significantly according to the chemical nature of the chosen film-forming polymer. At least one determining factor that may be used is the amount that is at least sufficient to confer the expected long-term stability, such as by way of an optimized rubbing resistance. Beyond this minimum amount, excess film-forming polymer may not be necessary. However, insofar as it does not prove detrimental to the expected qualities, there is no reason not to use this polymer in an amount greater than the minimum amount.

The at least one film-forming polymer may be present in the composition in a form chosen from liposoluble and water-soluble forms and in the form of particles dispersed in an aqueous medium and non-aqueous solvent medium.

Liposoluble Form

Examples of liposoluble polymers which may be mentioned are copolymers of a vinyl ester (the vinyl group being joined directly to the oxygen atom of the ester group, and the vinyl ester comprising a linear or branched, saturated hydrocarbon radical having 1 to 24 carbon atoms bonded to the carbonyl of the ester group) and at least one other monomer which can be a vinyl ester (different from the vinyl ester already present), an alkyl vinyl ether (wherein the alkyl group comprises from 2 to 24 carbon atoms) or an allyl or methallyl ester (comprising a linear or branched, saturated hydrocarbon radical having 1 to 24 carbon atoms bonded to the carbonyl of the ester group). These copolymers may be crosslinked with the aid of crosslinking agents, which may be either of the vinyl type or of the allyl or methallyl type, such as tetrallyloxyethane, divinylbenzene, divinyl octanedioate, divinyl dodecanedioate and divinyl octadecanedioate.

The following copolymers may be mentioned by way of example: vinyl acetate/vinyl laurate, vinyl propionate/allyl laurate, vinyl propionate/vinyl laurate, and allyl 2,2-dimethylpentanoate/vinyl laurate.

Liposoluble film-forming polymers which may also be mentioned may be liposoluble homopolymers, such as those resulting from the homopolymerization of vinyl esters having from 9 to 22 carbon atoms, or of alkyl acrylates or methacrylates, the alkyl radicals having from 2 to 24 carbon atoms.

The following may be mentioned as examples of liposoluble homopolymers: polyvinyl laurate and polylauryl (meth)acrylates, it being possible for these poly(meth)acrylates to be crosslinked with the aid of ethylene glycol dimethacrylate or tetraethylene glycol dimethacrylate.

The liposoluble copolymers and homopolymers defined above are known and are described, for example, in French Application No. FR-A-2 232 303; they can have a weight-average molecular weight ranging from 2000 to 500,000 and, for example, from 4000 to 200,000.

The following may also be mentioned as liposoluble film-forming polymers which may be used in the disclosure: polyalkylenes, for example, copolymers of C₂ to C₂₀ alkenes, such as polybutene, alkyl celluloses having a linear or branched, saturated or unsaturated C₁ to C₈ alkyl radical, such as ethyl cellulose and propyl cellulose, and copolymers of vinylpyrrolidone (VP), such as copolymers of vinylpyrrolidone and a C₂ to C₄₀ alkene, for example, a C₃ to C₂₀ alkene. The following may be mentioned as examples of VP copolymers which can be used in the disclosure: VP/vinyl acetate and VP/ethyl methacrylate copolymers, butylated polyvinylpyrrolidone (PVP), and VP/ethyl methacrylate/methacrylic acid, VP/eicosene, VP/hexadecene, VP/triacontene, VP/styrene, and VP/acrylic acid/lauryl methacrylate copolymers.

Dispersed Form

The at least one film-forming polymer may also be present in the composition in the form of particles dispersed in an aqueous phase, this dispersion generally being known as a latex or pseudolatex, in a non-aqueous solvent phase or liquid fatty phase. The techniques for preparing these dispersions are well known to those skilled in the art.

a) Aqueous Dispersion

Aqueous dispersions of the at least one film-forming polymer which may be used are the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by AVECIA-NEORESINS, “Dow Latex 432®” by DOW CHEMICAL and “Daitosol 5000 AD®” by DAITO KASEY KOGYO; the aqueous polyurethane dispersions sold under the names “Neorez R-981®” and “Neorez R-974®” by AVECIA-NEORESINS, “Avalure UR-405®”, “Avalure UR-410®”, “Avalure UR-425®”, “Avalure UR-450®”, “Sancure 875®”, “Sancure 861”, “Sancure 878®” and “Sancure 2060®” by GOODRICH, “Impranil 85®” by BAYER and “Aquamere H-1511®” by HYDROMER; the sulphopolyesters sold under the trade mark “Eastman AQ®” by EASTMAN CHEMICAL PRODUCTS; and vinylic dispersions such as “Mexomère PAM” from CHIMEX.

The at least one film-forming polymer may be, for example, homopolymers or graft or sequence block copolymers incorporating at least one block of a styrene polymer. For example, it may be possible to use copolymers comprising at least one block resulting from the polymerization of at least one ethylenic monomer having at least one optionally conjugated double bond, such as ethylene, butadiene or isoprene, and at least one block of a styrene polymer. If the ethylenic monomer comprises several optionally conjugated double bonds, the residual ethylenic units of unsaturation after polymerization may be generally hydrogenated. Thus, in known manner, the polymerization of isoprene leads, after hydrogenation, to the formation of an ethylene-propylene block and the polymerization of butadiene leads, after hydrogenation, to the formation of an ethylene-butylene block.

b) Non-Aqueous Dispersion

The at least one film-forming polymer used in this variant may be of any kind. Thus, it may be possible to employ a polymer chosen from a free radical polymer, a polycondensate and a polymer of natural origin, and mixtures thereof. The polymer may be chosen by those skilled in the art according to its properties.

One of the properties of this type of polymer dispersion in a composition of the disclosure may be the possibility of varying the glass transition temperature (Tg) of the polymer or polymer system (polymer plus additive of the plasticizer type) and thus, of changing from a soft polymer to a more or less hard polymer, making it possible to adjust the mechanical properties of the film obtained with the composition of the disclosure.

The polymers which may be used in the composition of the disclosure, for example, have a weight-average molecular weight ranging from 2000 to 10,000,000. The polymer may have a glass transition temperature ranging from −100° C. to 300° C. and, for example, from −10° C. to 50° C.

It may be possible to use filmifiable polymers that, for example, have a low glass transition temperature less than or equal to the temperature of the skin and especially less than or equal to about 40° C. As used herein, “filmifiable polymer” is understood as meaning a polymer that is capable of forming an isolatable film, either by itself or in the presence of a plasticizer. This gives a dispersion, which can filmify when applied to a substrate, which may not be the case when using mineral pigment dispersions according to the prior art.

If the polymer has a glass transition temperature that is too high for the desired application, then a plasticizer may be associated therewith so as to lower the glass transition temperature of the mixture used. The plasticizer may be chosen from those normally used in the field of application and, for example, from compounds capable of being solvents for the polymer.

Filmifiable polymers which may be mentioned are chosen from acrylic and vinylic, free radical homopolymers and copolymers such as having a Tg less than or equal to about 40° C. and, for example, ranging from −10° C. to 30° C., and mixtures thereof.

Non-filmifiable polymers which may be mentioned are chosen from vinylic and acrylic, free radical homopolymers and copolymers such having a Tg greater than about 40° C. and, for example, ranging from 45° C. to 100° C., and mixtures thereof. In association with the liquid fatty phase, the non-filmifiable polymer makes it possible to form a continuous and homogeneous deposit on eyelashes.

As used herein, free radical polymer is understood as meaning a polymer obtained by the polymerization of unsaturated monomers, such as those with ethylenic unsaturation, each monomer being capable of homopolymerizing (in contrast to polycondensates). The free radical polymers may be, for example, vinylic polymers or copolymers and, for example, acrylic polymers.

The vinylic polymers may result from the polymerization of ethylenically unsaturated monomers having at least one acid group, and/or esters of these acid monomers, and/or amides of these acids.

α,β-ethylenically unsaturated carboxylic acids, such as acrylic acid, methacrylic acid, crotonic acid, maleic acid and itaconic acid, may be used as monomers carrying acid groups. For example, other monomers that may be use are (meth)acrylic acid and crotonic acid and, further for example, (meth)acrylic acid.

The esters of acid monomers are, for example, chosen from (meth)acrylic acid esters (also called (meth)acrylates), such as alkyl (meth)acrylates wherein the alkyl is, for example, C₁-C₂₀ and, further for example, C₁-C₈, aryl (meth)acrylates wherein the aryl is such as C₆-C₁₀, and hydroxyalkyl (meth)acrylates such as the hydroxyalkyl is, for example, C₂-C₆.

Alkyl (meth)acrylates which may be mentioned, for example, are methyl (meth)acrylate, ethyl (meth)acrylate, butyl (meth)acrylate, isobutyl (meth)acrylate, 2-ethylhexyl (meth)acrylate, and lauryl (meth)acrylate.

Hydroxyalkyl (meth)acrylates which may be mentioned, for example, are hydroxyethyl (meth)acrylate and 2-hydroxypropyl (meth)acrylate.

Aryl (meth)acrylates which may be mentioned, for example, are benzyl acrylate and phenyl acrylate.

In one embodiment, (meth)acrylic acid esters are alkyl (meth)acrylates.

Copolymers of (meth)acrylic acid and an alkyl (meth)acrylate wherein the alkyl is, for example, C₁-C₄ may be used as the free radical polymers. Methyl acrylate or methacrylate/acrylic acid copolymers may be used.

Amides of the monomeric acids which may be used are (meth)acrylamides and, for example, N-alkyl(meth)acrylamides wherein the alkyl is C₂-C₁₂, such as N-ethylacrylamide, N-t-butylacrylamide and N-octylacrylamide; and N,N-dialkyl(C₁-C₄)(meth)-acrylamides.

The vinylic polymers may also result from the polymerization of ethylenically unsaturated monomers having at least one amine group, in the free form, in the partially or totally neutralized form or in the partially or totally quaternized form. Such monomers may be, e.g., dimethylaminoethyl (meth)acrylate, dimethylaminoethylmethacrylamide, vinylamine, vinylpyridine, and diallyldimethylammonium chloride.

The vinylic polymers may also result from the homopolymerization or copolymerization of at least one monomer chosen from vinyl esters and styrene monomers. For example, these monomers may be polymerized with acid monomers and/or their esters and/or their amides, such as those mentioned above.

Examples of vinyl esters which may be mentioned are vinyl acetate, vinyl propionate, vinyl neodecanoate, vinyl pivalate, vinyl benzoate, and vinyl t-butylbenzoate.

Styrene monomers which may be mentioned are styrene and alpha-methylstyrene.

The list of monomers given does not imply a limitation and it is possible to use any monomer known to those skilled in the art that falls in the categories of acrylic and vinylic monomers (including monomers modified with a silicone chain).

Other vinylic monomers which may also be mentioned are:

N-vinylpyrrolidone; vinylcaprolactam; vinyl-N-alkyl(C₁-C₆)pyrroles; vinyloxazoles; vinylthiazoles; vinylpyrimidines; and vinylimidazoles; and

olefins such as ethylene, propylene, butylene, isopropene, and butadiene.

The vinylic polymer may be crosslinked with the aid of a difunctional monomer, for example, one comprising at least two ethylenic units of unsaturation, such as ethylene glycol dimethacrylate or diallyl phthalate.

Without implying a limitation, the polymers of the disclosure may be chosen from the following polymers and copolymers: polyurethanes, acrylic polyurethanes, polyureas, polyureapolyurethanes, polyesterpolyurethanes, polyetherpolyurethanes, polyesters, polyesteramides and polyesters with an alkyl fatty chain; acrylic and vinylic polymers and copolymers; polyacrylamides; silicone polymers; fluorinated polymers; and mixtures thereof.

With such a dispersion of polymers particles, it may be possible to calibrate the size of the polymer particles at will and to modulate their size “polydispersity” during synthesis. It may, thus, be possible to obtain particles of very small size, which are invisible to the naked eye when they are in the composition. For example, the nanoparticles may have a size ranging from 5 nm to 600 nm, given that the particle dispersions become much less stable beyond about 600 nm, such as ranging from 50 nm to 250 nm.

The dispersed polymer may be generally used in an effective amount for depositing a film on keratin fibers that resists rubbing, optionally in the presence of water and/or sebum and/or perspiration.

In practice, the polymer dispersed in a liquid fatty phase may be present in an amount ranging from 2% to 50% by weight, such as from 4% to 40% by weight and, further for example, from 5% to 30% by weight, based on the total weight of the composition.

As used herein, “liquid fatty phase” is understood as meaning any non-aqueous medium that is liquid at room temperature (25° C.) and atmospheric pressure. This fatty phase may comprise a volatile liquid fatty phase and/or a non-volatile liquid fatty phase, each of the phases comprising at least one liquid oil.

As used herein, “liquid fatty phase” is understood as meaning any non-aqueous medium capable of evaporating from the skin in less than one hour. This volatile phase comprises, for example, oils having a vapor pressure ranging from 10⁻³ mmHg to 300 mmHg (0.13 Pa to 40,000 Pa) at room temperature and atmospheric pressure.

The liquid fatty phase wherein the polymer is dispersed comprises any physiologically acceptable and, for example, cosmetically acceptable oil chosen from carbon-comprising, hydrocarbon, fluorinated and silicone oils of mineral, animal, vegetable and synthetic origin, by themselves or in a mixture insofar as they form a homogeneous and stable mixture and are compatible with the envisaged use.

The total liquid fatty phase of the composition may comprise from 5% to 98% by weight and, for example, from 20% to 85% by weight, based on the total weight of the composition.

The following may, thus, be mentioned as liquid fatty phases which may be used in the disclosure, which are chosen from: hydrocarbon oils such as paraffin oil and petrolatum, mink, turtle and soya oil, perhydrosqualene, and sweet-almond, calophyllum, palm, grapeseed, sesame, maize, parleam, arara, colza, sunflower, cottonseed, apricot, castor, avocado, jojoba, olive and cereal germ oil; esters of lanolic acid, oleic acid, lauric acid and stearic acid; fatty esters such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyidecyl palmitate, 2-octyldodecyl myristate and lactate, 2-diethylhexyl succinate, diisostearyl malate, and glycerol or diglycerol triisostearate; higher fatty acids such as myristic acid, palmitic acid, stearic acid, behenic acid, oleic acid, linoleic acid, linolenic acid and isostearic acid; higher fatty alcohols such as cetanol, stearyl alcohol, oleyl alcohol, linoleyl and linolenyl alcohol, isostearyl alcohol and octyidodecanol; silicone oils such as polydimethylsiloxanes (PDMS) that are optionally phenylated, such as phenyltrimethicones, and optionally substituted by optionally fluorinated aliphatic and aromatic groups and by functional groups such as hydroxyl, thiol and amine groups; polysiloxanes modified by fatty acids, fatty alcohols and polyoxyalkylenes; fluorinated silicones; and perfluorinated oils.

In one embodiment, it may be possible to use at least one oil that may be volatile at room temperature. These volatile oils favor the production of a film with total “no transfer” properties, i.e. which is totally resistant to rubbing. Evaporation of these oils leaves a film-forming deposit that is flexible and non-sticky. These volatile oils may also facilitate application of the composition to keratin fibers such as eyelashes.

These volatile oils may be hydrocarbon oils or silicone oils optionally comprising alkyl or alkoxy groups that are pendent and at the end of the silicone chain.

Linear or cyclic silicones having from 2 to 7 silicon atoms may be mentioned as volatile silicone oils which may be used in the disclosure, these silicones optionally comprising alkyl or alkoxy groups having from 1 to 10 carbon atoms. Octamethylcyclotetra-siloxane, decamethylcyclopentasiloxane, hexadecamethylcyclohexasiloxane, heptamethyl-hexyltrisiloxane, and heptamethyloctyltrisiloxane may be mentioned, for example.

Volatile hydrocarbon oils, which may be mentioned, are C₈-C₁₆ isoparaffins such as ISOPARs®, PERMETYLs® and, for example, isododecane.

These volatile oils may be present in the composition in an amount ranging from 5% to 97.99% and, for example, from 30% to 75% of the total weight of the composition.

In one embodiment of the disclosure, the liquid fatty phase is chosen from the group comprising:

non-aqueous liquid compounds having a global solubility parameter according to the HANSEN solubility space that is less than 17 (MPa)^{1/2},

monoalcohols having a global solubility parameter according to the HANSEN solubility space that is less than or equal to 20 (MPa)^{1/2}, and

mixtures thereof.

The polymer dispersion may be manufactured as described in document European Patent No. EP-A-749 747. Polymerization may be effected in dispersion, i.e. by precipitating the polymer as it is formed, the particles formed being protected with a stabilizer.

A mixture is therefore prepared which comprises the initial monomers and a free radical initiator. This mixture is dissolved in a solvent referred to in the remainder of the present description as “synthesis solvent”. If the fatty phase is a non-volatile oil, it may be possible to effect polymerization in an apolar organic solvent (synthesis solvent), then add the non-volatile oil (which has to be miscible with the synthesis solvent) and selectively distil the synthesis solvent.

The chosen synthesis solvent is, therefore, such that the initial monomers and the free radical initiator may be soluble therein and the particles of polymer obtained are insoluble therein so that they precipitate as they are formed. For example, the synthesis solvent may be chosen from alkanes such as heptane, isododecane, and cyclohexane.

If the chosen fatty phase is a volatile oil, then polymerization may be effected directly in the oil, which therefore also acts as synthesis solvent. The monomers and the free radical initiator should also be soluble therein and the polymer obtained should be insoluble therein.

The monomers are, for example, present in the synthesis solvent, before polymerization, in an amount ranging from 5% to 20% of the weight of the reaction mixture. It may be possible for all of the monomers to be present in the solvent before the start of the reaction, or for part of the monomers to be added as the polymerization reaction proceeds.

The free radical initiator may be, for example, azobisisobutyronitrile or 2-tert-butylperoxyethyl hexanoate.

The polymer particles may be stabilized on the surface in the course of polymerization through a stabilizer, which may be a sequence polymer, a graft polymer and/or a random polymer, by itself or in a mixture. Stabilization may be effected by any known means and, for example, by adding the sequence polymer, graft polymer and/or random polymer directly during polymerization.

The stabilizer is, for example, also present in the mixture before polymerization. However, it may also be possible to add it continuously, for example, if the monomers are also added continuously.

It may be possible to use from 2% to 30% by weight and, for example, from 5% to 20% by weight of stabilizer, based on the initial mixture of monomers.

If a graft and/or sequence polymer is used as the stabilizer, then the synthesis solvent may be chosen in such a way that at least part of the grafts or sequences of the stabilizing polymer is soluble in the solvent, the other part of the grafts or sequences being insoluble therein. The stabilizing polymer used during the polymerization should be soluble or dispersible in the synthesis solvent. Furthermore, a stabilizer whose insoluble sequences or grafts have a certain affinity for the polymer formed during polymerization may be chosen.

Graft polymers which may be mentioned are silicone polymers grafted with a hydrocarbon chain and hydrocarbon polymers grafted with a silicone chain.

Graft copolymers having, e.g., an insoluble backbone of the polyacrylic type with soluble grafts of the poly(12-hydroxystearic acid) type may also suitable.

Graft or sequence block copolymers comprising at least one block of the polyorganosiloxane type and at least one block of a free radical polymer, such as graft copolymers of the acrylic/silicone type, which may be employed, for example, if the non-aqueous medium is a silicone medium, may be used.

The stabilizer may be chosen from graft and sequence block copolymers comprising at least one block of the polyorganosiloxane type and at least one polyether. The polyorganopolysiloxane block may be, for example, a polydimethylsiloxane or a polyalkyl(C₂-C₁₈)methylsiloxane, and the polyether block may be a poly-C₂-C₁₈-alkylene, such as polyoxyethylene and/or polyoxypropylene. Dimethicone copolyols or alkyl(C₂-C₁₈)methicone copolyols may be used, for example, it being possible to use the dimethicone copolyol sold under the name “DOW CORNING 3225C” by DOW CORNING or the laurylmethicone copolyol sold under the name “DOW CORNING Q2-5200” by “DOW CORNING”.

Graft or sequence block copolymers, which may be used, are copolymers comprising at least one block resulting from the polymerization of at least one ethylenic monomer having at least one optionally conjugated double bonds, such as ethylene, butadiene or isoprene, and at least one block of a styrene polymer. If the ethylenic monomer comprises several optionally conjugated double bonds, the residual ethylenic units of unsaturation after polymerization may also be hydrogenated. Thus, in a known manner, the polymerization of isoprene leads, after hydrogenation, to the formation of an ethylene-propylene block and the polymerization of butadiene leads, after hydrogenation, to the formation of an ethylene-butylene block. The following may be mentioned among these sequence copolymers: “diblock” or “triblock” copolymers of the polystyrene/polyisoprene or polystyrene/polybutadiene type, such as those sold under the name “LUVITOL HSB” by BASF, of the polystyrene/copoly(ethylene-propylene) type, such as those sold under the name “KRATON” by Shell Chemical Co., or of the polystyrene/copoly(ethylene-butylene) type.

Polymethyl methacrylate/polyisobutylene bisequence or trisequence copolymers, or graft copolymers with a polymethyl methacrylate backbone and polyisobutylene grafts, may be mentioned as graft or sequence block copolymers comprising at least one block resulting from the polymerization of at least one ethylenic monomer, such as ethylene or isobutylene, and at least one block of an acrylic polymer such as methyl methacrylate.

Polyoxyethylene/polybutadiene or polyoxyethylene/polyisobutylene bisequence or trisequence copolymers may be mentioned as graft or sequence block copolymers comprising at least one block resulting from the polymerization of at least one ethylenic monomer, and at least one block of a polyether, such as a C₂-C₁₈ polyoxyalkylene, for example, polyoxyethylene and/or polyoxypropylene.

It may also be possible to employ copolymers of C₁-C₄-alkyl (meth)acrylates and C₈-C₃₀-alkyl (meth)acrylates. The stearyl methacrylate/methyl methacrylate copolymer may be mentioned, for example.

If a random polymer is used as the stabilizer, then it may be chosen so as to possess a sufficient amount of groups to render it soluble in the envisaged synthesis solvent.

If the synthesis solvent is apolar, then the chosen stabilizer may, for example, be a polymer that covers the particles as completely as possible, several chains of stabilizing polymers being adsorbed onto one particle of the polymer obtained by polymerization.

The stabilizer used in this case is, for example, either a graft polymer or a sequence polymer so as to improve the interfacial activity. For example, sequences or grafts that may be insoluble in the synthesis solvent cover the surface of the particles more voluminously.

If the liquid synthesis solvent comprises at least one silicone oil, then the stabilizer may be, for example, chosen from a group comprising graft and sequence block copolymers comprising at least one block of the polyorganosiloxane type and at least one block of a free radical polymer, a polyether or a polyester, such as polyoxy(C₂-C₁₈)alkylene blocks and, for example, polyoxypropylene and/or polyoxyethylene blocks.

If the liquid fatty phase does not comprise silicone oil, then the stabilizer may be, for example, chosen from the group comprising:

(a) graft and sequence block copolymers comprising at least one block of the polyorganosiloxane type and at least one block of a free radical polymer, a polyether and a polyester,

(b) copolymers of C₁-C₄-alkyl acrylates and methacrylates, and C₈-C₃₀-alkyl acrylates and methacrylates,

(c) graft and sequence block copolymers comprising at least one block resulting from the polymerization of at least one ethylenic monomer with conjugated double bonds, and at least one block of a vinylic or acrylic polymer, a polyether and a polyester, and mixtures thereof.

Diblock polymers as the stabilizer may be used.

Methyl acrylate or methacrylate/acrylic acid copolymer stabilized on the surface, in isododecane, with a polystyrene/copoly(ethylene-propylene) sequence diblock copolymer may be used.

If appropriate, the composition according to the disclosure may also comprise a plasticizer to favor the formation of a film with the film-forming polymer. Such a plasticizer may be chosen from all the compounds known to those skilled in the art as being capable of fulfilling the desired function.

### Polymer(s) Soluble in the Solvent Medium and Having at Least One Crystallizable Part

The composition according to the disclosure comprises at least one additional polymer that is soluble in the solvent medium, such as non-aqueous solvent medium, of the composition according to the disclosure, and has at least one crystallizable part.

As used herein, “polymer soluble in the solvent medium” is understood as meaning a polymer which, when introduced by itself in an amount greater than at least 0.01% by dry weight, and in an amount corresponding to that envisaged for the desired final composition, is soluble in the solvent medium at room temperature, generally in the order of 25° C., and at atmospheric pressure (750 mmHg, i.e. 10⁵ Pa).

As stated above, this additional polymer may be different from the chosen film-forming polymer in the composition according to the disclosure.

In terms of the present disclosure, the word “polymer” is understood as meaning a compound possessing at least two repeat units, such as at least three repeat units, for example, at least ten repeat units or at least fifteen repeat units. The polymer according to the disclosure may be generally comprised of at least two repeat units of a different nature (copolymer). The polymers used in the disclosure are generally of synthetic origin and are characterized by molecular weights ranging from 200 to 1,000,000 g/mol, such as from 500 to 500,000 g/mol and, for example, from 1000 to 300,000 g/mol.

For example, the additional polymers used in the present disclosure may be copolymers that are solubilized and non-crystallized in the medium at room temperature, and necessarily comprise at least one crystallizable part, A, and at least one so-called amorphous, non-crystallizable part, B.

Apart from this specific structure, they, for example, possess both an affinity for the waxes by virtue of the part A and an affinity for the solvent by virtue of the part B, so they effectively participate in the dispersion of the waxes in the solvent medium, such as non-aqueous solvent medium.

The crystallizable part of the polymers used in the present disclosure are present in an amount of at least 5%, such as at least 10% and at most 50% and, further for example, are present in an amount ranging from 30% to 50% by weight, based on the total weight of each polymer.

The crystallizable part A of a copolymer according to the disclosure may comprise a pendent chain bonded to the backbone of the polymer and/or a sequence integrated directly into this backbone and/or at least one terminal chain. These copolymers may have any chemical structure: random, sequence, graft copolymers, and/or dendrimers.

Likewise, the amorphous part of a copolymer according to the disclosure may comprise a pendent chain bonded to the backbone of the copolymer and/or a sequence integrated directly into this backbone and/or at least one terminal chain.

The words or expressions given below are defined in terms of the disclosure:

“crystallizable part A”: a concatenation of at least 5 repeat units such that the homopolymer corresponding to this repeat unit would be characterized by a degree of crystallinity of more than 30%,

“amorphous part B”: a concatenation of at least 5 repeat units such that the homopolymer corresponding to this repeat unit would be characterized by a degree of crystallinity of less than 5%, or zero,

“sequence integrated into the backbone”: a group of atoms comprising the repetition of a monomer unit and forming part of the main polymer chain,

“pendent chain or side group”: a group of atoms comprising a branch on the polymer backbone, and

“terminal chain”: a group of atoms located at one or both ends of the backbone.

a) Random Copolymers

The random copolymers are, for example, polymers with crystallizable pendent chains which comprise units resulting from the polymerization of at least two monomers, at least one of which has a crystallizable hydrophobic side chain, X, which may be represented by formula I: 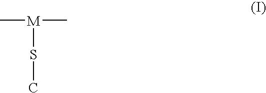
wherein M is an atom of the polymer backbone, S is a spacer, and C is a crystallizable group.

The crystallizable chains “—S—C” may be chosen form aliphatic and aromatic, linear, branched and cyclic, and optionally fluorinated or perfluorinated. “S” is, for example, a linear, branched or cyclic group (CH₂)ₙ, (CH₂CH₂O)ₙ or (CH₂O), where n is an integer ranging from 0 to 22. In one embodiment, “S” is a linear group and “S” and “C” are different.

If the crystallizable chains “—S—C” are aliphatic hydrocarbon chains, then they comprise alkyl hydrocarbon chains having at least 11 carbon atoms, at most 40 carbon atoms and, for example, at most 24 carbon atoms. For example, they are aliphatic chains or alkyl chains having at least 12 carbon atoms, such as C₁₂-C₂₄ alkyl chains. If they are fluorinated or perfluorinated alkyl chains, then they comprise at least 6 fluorinated carbon atoms and, for example, at least 11 carbon atoms of which at least 6 are fluorinated.

Examples which may be mentioned of polymers with at least one crystallizable pendent chains are those comprising units resulting from the polymerization of at least one of the following monomers: saturated alkyl (meth)acrylates wherein the alkyl group is C₁₂-C₂₄, perfluoroalkyl (meth)acrylates wherein the perfluoroalkyl group is C₁₂-C₁₅, N-alkyl(meth)acrylamides wherein the alkyl group is C₁₂ to C₂₄, with or without a fluorine atom, vinyl or allyl esters with alkyl or perfluoroalkyl chains wherein the alkyl group is C₁₂ to C₂₄ (with at least 6 fluorine atoms per perfluoroalkyl chain), vinyl ethers with alkyl or perfluoroalkyl chains wherein the alkyl group is C₁₂ to C₂₄, and with at least 6 fluorine atoms per perfluoroalkyl chain, C₁₂ to C₂₄ alpha-olefins, e.g., octadecene, para-alkylstyrenes wherein the alkyl group contains from 12 to 24 carbon atoms, and mixtures thereof.

The following may be mentioned by way of illustration of these polymers which may be used in the present disclosure: copolymers of saturated linear C₁₂ to C₃₀ alkyl (meth)acrylates comprising the crystallizable part A, and linear C₄ to C₁₀ or branched or cyclic and/or unsaturated C₄ to C₃₀ alkyl (meth)acrylates comprising the amorphous part B.

The following may be mentioned, for example, among the copolymers of vinyl esters with saturated linear C₁₂ to C₃₀ alkyl groups comprising the crystallizable part A, and vinyl esters with linear C₄ to C₁₀ or branched or cyclic and/or unsaturated C₄ to C₃₀ alkyl groups comprising the amorphous part B: copolymers of vinyl acetate and vinyl stearate or allyl stearate, such as the allyl stearate/vinyl acetate copolymer sold under the name “Mexomère PQ®” by CHIMEX.

If the polymers result from a polycondensation, the crystallizable hydrocarbon and/or fluorinated chains as defined above are carried by a monomer, which may be a diacid, a diol, a diamine or a diisocyanate.

b) Sequence Copolymers

These copolymers comprise at least two types of sequences of a different chemical nature, one of which is crystallizable and comprises the part A. In the case of the sequence copolymers, at least one of the amorphous sequences B should be soluble in the medium.

Examples which may be mentioned are:

olefin or cycloolefin sequence copolymers with a crystallizable chain, such as those derived from the sequence polymerization of:

cyclobutene, cyclohexene, cyclooctene, norbornene (i.e., bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof, with

ethylene, propylene, 1-butene, 3-methyl-1-butene, 1-hexene, 4-methyl-1-pentene, 1-octene, 1-decene, 1-eicosene or mixtures thereof,

the hydrogenated polybutylene terephthalate/polyisoprene sequence or multisequence block copolymers cited in the article “Study of morphological and mechanical properties of PP/PBT” by B. Boutevin et al., Polymer Bulletin, 34, 117-123 (1995),

the polyethylene/copoly(ethylene-propylene) sequence block copolymers cited in the article “Morphology of semi-crystalline block copolymers of ethylene-(ethylene-alt-propylene)” by P. Rangarajan et al., Macromolecules, 26, 4640-4645 (1993), and in the article “Polymer aggregates with crystalline cores: the system poly(ethylene)-poly(ethylene-propylene)” by P. Richter et al., Macromolecules, 30, 1053-1068 (1997), and

the polyethylene/polyethylethylene sequence block copolymers cited in the general article “Crystallization in block copolymers” by l. W. Hamley, Advances in Polymer Science, vol. 148, 113-137 (1999).

These polymers may have a single crystallizable sequence or a repetition of crystallizable sequences. In the latter case, these crystallizable sequences may be of an identical or different chemical nature.

c) Copolymers with At Least One Terminal Crystallizable Sequences

The following may be mentioned as examples in this category:

polycondensates of the polyamide type resulting from condensation between (α) at least one acid chosen from dicarboxylic acids having at least 32 carbon atoms, such as dimeric fatty acids, and (β) an alkylenediamine, such as ethylenediamine, wherein the polyamide polymer comprises at least one terminal carboxylic acid group esterified or amidified with at least one monoalcohol or monoamine having from 12 to 30 saturated linear carbon atoms, and, for example, ethylenediamine/stearyl dilinoleate copolymers such as the one marketed under the name “Uniclear 100 VG®” by ARIZONA CHEMICAL; and

lipophilic polyester polycondensates whose ends are esterified with a crystallizable acid or alcohol comprising a saturated linear C₁₂ to C₃₀ carbon chain, such as poly(12-hydroxystearic acid) wherein at least one end is esterified with stearic acid, for example, “Solsperse 21000®” marketed by AVECIA.

Ethylene/vinyl acetate copolymers, ethylene/maleic anhydride copolymers, hydrogenated butadiene/isoprene sequence copolymers and ethylene/maleic anhydride/vinyl acetate terpolymers may be mentioned, for example, as complementary illustrations of the copolymers according to the disclosure.

According to one embodiment of the present disclosure, the polymer that is soluble in the solvent medium, such as non aqueous solvent medium, and has at least one crystallizable part, or a mixture of such polymers, may be present in the composition according to the disclosure in a portion equal or greater than 0.5% by weight, based on the total weight of the composition.

According to another embodiment of the present disclosure, the polymer that may be soluble in the solvent medium, such as non-aqueous solvent medium, and has at least one crystallizable part, or a mixture of such polymers, may be present in the composition according to the disclosure in a proportion ranging from 0.01% to 30%, such as from 0.1% to 20% and, for example, from 0.5% to 10% by weight, based on the total weight of the composition.

Polymer with at Least One Styrene Unit

These polymers are, for example, homopolymers or graft or sequence block copolymers incorporating at least one block of a styrene polymer.

For example, it may be possible to use copolymers comprising at least one block resulting from the polymerization of at least one ethylenic monomer having at least one optionally conjugated double bonds, such as ethylene, butadiene or isoprene, and at least one block of a styrene polymer. If the ethylenic monomer comprises several optionally conjugated double bonds, the residual ethylenic units of unsaturation after polymerization are generally hydrogenated. Thus, in known manner, the polymerization of isoprene leads, after hydrogenation, to the formation of an ethylene-propylene block and the polymerization of butadiene leads, after hydrogenation, to the formation of an ethylene-butylene block.

As stated above, this polymer with at least one styrene unit may be different from the film-forming polymer chosen according to the disclosure.

In one embodiment, this polymer may be identical to that used as the stabilizer for a non-aqueous dispersion of the at least one film-forming polymer as defined above.

As used herein, “diblock” or “triblock” sequence copolymers of the polystyrene/polyisoprene or polystyrene/polybutadiene type, such as those marketed under the name “Luvitol HSB®” by BASF, of the polystyrene/copoly(ethylene-propylene) type, such as those marketed under the name “Kraton®” by SHELL CHEMICAL CO., or of the polystyrene/copoly(ethylene-butylene) type may be suitable for the disclosure.

This type of copolymer with styrene units may be present in an amount ranging from 0.01% to 30%, such as from 0.1% to 20% and, for example, from 0.5% to 10% by weight, based on the total weight of the composition.

As a non-limiting illustration of a composition according to the disclosure, those comprising at least:

from 20% to 50% by weight of at least one film-forming polymer, such as a non-aqueous dispersion of particles and, for example, poly(methyl methacrylate/acrylic acid) stabilized on the surface, in isododecane, with a polystyrene/copoly(ethylene-propylene) sequence diblock copolymer,

from 0.1% to 10% by weight of at least one polymer with styrene units, for example, a polymer of the polystyrene/copoly(ethylene-propylene) type or an allyl stearate/vinyl acetate copolymer, and

from 0.1% to 10% of at least one polymer with a crystallizable part, such as poly(12-hydroxystearic acid) of which at least one end is esterified with stearic acid, may be mentioned, for example.

Wax(ES)

The compositions according to the disclosure comprises at least one wax in an amount greater than 3% by weight, based on the total weight of the composition.

The at least one wax considered within the framework of the present disclosure may be generally a lipophilic compound that is solid at room temperature (25° C.), has a reversible solid/liquid change of state, and has a melting point greater than or equal to 30° C. and capable of ranging up to 200° C. and, for example, up to 120° C.

By bringing the at least one wax to the liquid state (melting), it may be possible to render it miscible with the oils and to form a microscopically homogeneous mixture, but when the mixture is brought back to room temperature, the at least one wax recrystallizes in the oils of the mixture.

For example, the waxes suitable for the disclosure may have a melting point greater than or equal to 45° C. and, further for example, greater than or equal to 55° C.

In terms of the disclosure, the melting point corresponds to the temperature of the most endothermic peak observed in thermal analysis (DSC), as described in standard ISO 11357-3, 1999. The melting point of the at least one wax may be measured using a differential scanning calorimeter (DSC), e.g., the calorimeter sold under the name “MDSC 2920” by TA Instruments.

The measurement protocol is as follows:

A 5 mg sample of wax is placed in a crucible and subjected to a first temperature rise from −20° C. to 100° C. at a heating rate of 10° C./minute, then cooled from 100° C. to −20° C. at a cooling rate of 10° C./minute, and finally subjected to a second temperature rise from −20° C. to 100° C. at a heating rate of 5° C./minute. During the second temperature rise, the variation in the difference in power absorbed by the empty crucible and by the crucible containing the wax sample is measured as a function of temperature. The melting point of the compound is the temperature value that corresponds to the apex of the peak of the curve representing the variation in the difference in power absorbed as a function of temperature.

The at least one wax which may be used in the compositions according to the disclosure may be chosen from waxes of animal, vegetable, mineral and synthetic origin that are solid at room temperature, and mixtures thereof.

The waxes, which may be used in the compositions according to the disclosure, generally have a hardness ranging from 0.01 MPa to 15 MPa, such as of more than 0.05 MPa and, for example, of more than 0.1 MPa.

The hardness may be determined by measuring the compression force at 20° C. using the texture analyser sold under the name “TA-TX2i®” by RHEO, which is equipped with a stainless steel mobile in the form of a cylinder of diameter 2 mm, the change in the force (compression force or stretching force) (F) being measured as a function of time during the following operation:

The mobile is displaced at a speed of 0.1 mm/s and then penetrates the wax to a penetration depth of 0.3 mm. When the mobile has penetrated the wax to a depth of 0.3 mm, the mobile is kept stationary for 1 second (corresponding to the relaxation time) and is then withdrawn at a speed of 0.1 mm/s. During the relaxation time, the force (compression force) decreases sharply until it becomes zero; then, when the mobile is withdrawn, the force (stretching force) becomes negative and then increases again to the value 0. The hardness corresponds to the maximum compression force measured between the surface of the mobile and the wax at the moment when they are brought into contact. The value of this force is expressed in MPa.

To perform the hardness measurement, the wax is melted at a temperature equal to the melting point of the wax +20° C. The molten wax is poured into a receptacle of diameter 30 mm and depth 20 mm. The wax is recrystallized at room temperature (25° C.) for 24 hours and then kept for at least 1 hour at 20° C. before the hardness is measured.

The following may be mentioned, for example, by way of illustration at least one wax suitable for the disclosure: hydrocarbon waxes such as beeswax, lanolin wax, Chinese waxes, sumac wax, paraffins, certain polyethylene waxes, and waxy copolymers, as well as their esters.

It may be also possible to mention waxes obtained by the catalytic hydrogenation of animal or vegetable oils having linear or branched C₈-C₃₂ fatty chains. The following may be mentioned, for example, among these oils: isomerized jojoba oil, such as the trans-isomerized, partially hydrogenated jojoba oil manufactured or marketed by DESERT WHALE under the trade reference “Iso-Jojoba-50®”, hydrogenated sunflower oil, hydrogenated castor oil, hydrogenated copra oil, hydrogenated lanolin oil, and the di(1,1,1-trimethylolpropane) tetrastearate sold under the name “Hest 2T-4S®” by HETERENE.

Silicone waxes and fluorinated waxes may also be mentioned.

It may be also possible to use the waxes obtained by the hydrogenation of castor oil esterified with cetyl alcohol which are sold under the names “Phytowax ricin 16L64®” and “22L73®” by SOPHIM. Such waxes are described in French Patent Application FR-A-2 792 190.

In the present disclosure, it may be also possible to use waxes supplied in the form of small particles having a size in the order ranging from 0.5 micrometers to 30 micrometers, such as from 1 micrometer to 20 micrometers and, for example, from 5 micrometers to 10 micrometers, which are designated hereafter by the expression “microwaxes”. For distinguishing purposes, the waxes used according to the disclosure in the form of fragments of larger size are designated hereafter by the expression “waxes of traditional type”.

The following may be mentioned, for example, as microwaxes which may be used in the compositions according to the disclosure: carnauba microwaxes such as that marketed under the name “MicroCare 350®” by MICRO POWDERS, microwaxes of synthetic wax, such as that marketed under the name “MicroEase 114S®” by MICRO POWDERS, microwaxes comprising a mixture of carnauba wax and polyethylene wax, such as those marketed under the names “MicroCare 300®” and “310®” by MICRO POWDERS, microwaxes comprising a mixture of carnauba wax and synthetic wax, such as that marketed under the name “MicroCare 325®” by MICRO POWDERS, polyethylene microwaxes such as those marketed under the names “Micropoly 200®”, “220®”, “220L®” and “250S®” by MICRO POWDERS, and polytetrafluoroethylene microwaxes such as those marketed under the names “Microslip 519®” and “519L®” by MICRO POWDERS.

Some of the microwaxes mentioned above, for example, carnauba microwax, the microwax of synthetic wax “MicroEase 114S®” or the microwax comprising a mixture of carnauba wax and synthetic wax “MicroCare 325®”, have an incipient melting point greater than or equal to 45° C.

In the composition according to the disclosure, it may of course possible to use a mixture of waxes and, for example, to use at least one wax of traditional type such as, for example, a sticky wax and/or a wax having an incipient melting point greater than or equal to 45° C., and at least one wax known as microwaxes.

The composition according to the disclosure generally comprises from 10% to 70% by weight of waxes. For example, it may comprise from 15% to 65%, such as from 20% to 60% or from 25% to 55% by weight of the at least one wax, based on the total weight of the composition.

The at least one wax is present in the compositions according to the disclosure in the form of a dispersion of particles in the solvent medium, such as non-aqueous solvent medium.

The wax particles may have a variety of shapes, such as a substantially spherical shape.

Observation of a sample of the composition under a microscope at room temperature shows a good dispersion of the wax particles in the medium, with little or no aggregation of these particles, or a substantially identical distribution of the particles in all directions.

Colorant

The composition according to the disclosure may also comprise at least one colorant such as pulverulent materials, liposoluble colorants, and water-soluble colorants.

The pulverulent colorants may be chosen from pigments and nacres.

The pigments may be white or colored, mineral and/or organic and coated or uncoated. Mineral pigments, which may be mentioned, are titanium dioxide optionally treated on the surface, zirconium, zinc and cerium oxides, iron and chromium oxides, manganese violet, ultramarine blue, chromium hydrate, and ferric blue. Organic pigments, which may be mentioned, are carbon black, pigments of the D&C type, and lakes based on cochineal carmine, barium, strontium, calcium or aluminium.

The nacres may be chosen from white nacreous pigments such as mica coated with titanium or with bismuth oxychloride, colored nacreous pigments such as titanium mica with iron oxides, titanium mica with ferric blue or chromium oxide, for example, or titanium mica with an organic pigment of the aforementioned type, and nacreous pigments based on bismuth oxychloride.

Examples of liposoluble colorants are Sudan red, D&C Red 17, D&C Green 6, β-carotene, soya oil, Sudan brown, D&C Yellow 11, D&C Violet 2, D&C Orange 5, quinoline yellow, and annatto.

These colorants may be present in an amount ranging from 0.01% to 30% by weight, based on the total weight of the composition.

Fillers

The composition according to the disclosure may also comprise at least one filler.

The at least one filler may be chosen from the ones well known to those skilled in the art and commonly used in cosmetic compositions. The at least one filler may be mineral or organic and lamellar or spherical. The following may be mentioned: talcum, mica, silica, kaolin, powders of a polyamide such as Nylon® marketed under the name “Orgasol®” by ATOCHEM, poly-β-alanine and polyethylene, powders of tetrafluoroethylene polymers such as Teflon®, lauroyllysine, starch, boron nitride, expanded hollow polymer microspheres such as those of polyvinylidene chloride/acrylonitrile, e.g., those marketed under the name “Expancel®” by NOBEL INDUSTRIE, acrylic powders such as those marketed under the name “Polytrap®” by DOW CORNING, polymethyl methacrylate particles and silicone resin microbeads (for example “Tospearls®” from TOSHIBA), precipitated calcium carbonate, magnesium carbonate and hydrocarbonate, hydroxyapatite, hollow silica microspheres (“Silica Beads®” from MAPRECOS), glass or ceramic microcapsules, and metal soaps derived from carboxylic organic acids having from 8 to 22 carbon atoms, such as from 12 to 18 carbon atoms and, for example, zinc, magnesium or lithium stearate, zinc laurate or magnesium myristate.

The at least one filler may be present in an amount ranging from 0.1% to 25% and, for example, from 1% to 20% by weight, based on the total weight of the composition.

The composition of the disclosure may also comprise any cosmetically acceptable additive selected, for example, from those normally used in cosmetics, such as antioxidants, preservatives, perfumes, neutralizers, plasticizers, thickeners or gelling agents, fibers, cosmetic active ingredients, and mixtures thereof.

The gelling agents, which may be used in the compositions according to the disclosure, are generally lipophilic and may be organic or mineral and polymeric or molecular.

Mineral lipophilic gelling agents which may be mentioned are optionally modified clays such as hectorites modified with a C₁₀ to C₂₂ fatty acid ammonium chloride, like hectorite modified with distearyldimethylammonium chloride, for example, the one marketed under the name “Bentone 38V®” by ELEMENTIS.

Pyrogenic silica optionally hydrophobized on the surface and having a particle size below 1 μm may also be mentioned. It may be possible to modify the surface of the silica chemically through a chemical reaction that reduces the number of silanol groups present on the surface of the silica. For example, it may be possible to replace silanol groups with hydrophobic groups to give a hydrophobic silica. The hydrophobic groups may be:

trimethylsiloxy groups, which are obtained, for example, by treating pyrogenic silica in the presence of hexamethyldisilazane. Silicas treated in this way are called silica silylates according to CTFA (6th edition, 1995). They are marketed, e.g., under the references “Aerosil R812®” by DEGUSSA, and “CAB-O-SIL TS-530®” by CABOT.

dimethylsilyloxy or polydimethylsiloxane groups, which are obtained, for example, by treating pyrogenic silica in the presence of polydimethylsiloxane or dimethyldichlorosilane. Silicas treated in this way are called silica dimethyl silylates according to CTFA (6th edition, 1995). They are marketed, e.g., under the references “Aerosil R972®” and “Aerosil R974®” by DEGUSSA and “CAB-O-SIL TS-610®”, and “CAB-O-SIL TS-720®” by CABOT.

For example, the particle size of the hydrophobic pyrogenic silica may be nanometric to micrometric, ranging, e.g., from about 5 nm to 200 nm.

Examples of polymeric organic lipophilic gelling agents are partially or totally crosslinked elastomeric organopolysiloxanes of three-dimensional structure, such as those marketed under the names “KSG6®”, “KSG16®” and “KSG18®” by SHIN-ETSU, “Trefil E-505C®” and “Trefil E-506C®” by DOW-CORNING, “Gransil SR-CYC®”, “SR DMF10®”, “SR-DC556®”, “SR 5CYC gel®”, “SR DMF 10 gel®” and “SR DC 556 gel®” by GRANT INDUSTRIES, and “SF 1204®” and “JK 113®” by GENERAL ELECTRIC; ethyl cellulose such as that sold under the name “Ethocel®” by DOW CHEMICAL; and galactomannans comprising from one to six and, for example, from two to four hydroxyl groups per se and substituted by a saturated or unsaturated alkyl chain, such as guar gum alkylated by C₁ to C₆ and, for example, C₁ to C₃ alkyl chains, and mixtures thereof.

Fatty acid dextrin esters such as dextrin palmitates, such as those marketed under the name “Rheopearl TL®” or “Rheopearl KL®” by CHIBA FLOUR, may also be mentioned among the gelling agents which may be used in the compositions according to the disclosure.

The composition according to the disclosure may also comprise fibers to improve the elongating effect.

As used herein, “fiber” should to be understood as meaning an object of length L and diameter D which is such that L is very much greater than D, D being the diameter of the circle in which the cross-section of the fiber is inscribed. For example, the ratio L/D (or form factor) may be chosen from a range from 3.5 to 2500, such as from 5 to 500, and, for example, from 5 to 150.

The fibers may be present in the composition according to the disclosure in an amount ranging from 0.01% to 10% by weight, such as from 0.1% to 5% by weight, and, for example, from 0.3% to 3% by weight, based on the total weight of the composition.

The following may be mentioned, for example, as cosmetic active ingredients which may be used in the compositions according to the disclosure: emollients, moisturizers, vitamins, and filters, such as sun filters.

Of course, those skilled in the art will take care to choose any complementary additives and/or their amount in such a way that the advantageous properties of the composition according to the disclosure are unaffected or substantially unaffected by the envisaged addition.

The present disclosure further relates to a method of making up keratin fibers wherein a composition as defined above is applied to the keratin fibers, such as eyelashes.

For example, the compositions of the disclosure may be applied to eyelashes using a brush or comb.

Other than in the operation examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that numerical ranges and parameters setting forth the broad scope of the disclosure are approximations, the numerical values set forth in the specific examples are reported to as precisely as possible. Any numerical value, however, inherently contains certain errors necessarily resulting from the standard deviation found in their respective testing measurements.

The Examples, which follow, are given by way of illustration of the disclosure and without implying a limitation.

### EXAMPLE

The compositions of the test formulations are shown in Table I below.

TABLE I

| | Comparative | Comparative | Comparative | | |
|---|---|---|---|---|---|
| | Formulation | Formulation | Formulation | | |
| | No. 1 | No. 2 | No. 3 | F1 | F2 |
| Carnauba wax | 4.5 | 4.7 | 4.7 | 4.7 | 4.7 |
| Beeswax | 8.3 | 13.7 | 13.7 | 13.7 | 13.7 |
| Polyolefin wax (Performa V260 | — | 0.6 | 0.6 | 0.6 | 0.6 |
| from New Phase Technologies) | | | | | |
| Paraffin wax | 2.2 | — | — | — | — |
| Polybutylene (Indopol H-1500 | — | 3 | 3 | 3 | 3 |
| from AMOCO) | | | | | |
| Stearate of poly(12- | — | — | 0.6 | 0.6 | 0.6 |
| hydroxystearic acid) oligomer | | | | | |
| (Solsperse 21000 from | | | | | |
| AVECIA) | | | | | |
| Vinyl acetate/allyl stearate | 2.2 | 0.75 | 0.75 | 0.75 | 0.75 |
| copolymer (Mexomere PQ from | | | | | |
| CHIMEX) | | | | | |
| Polyvinyl laurate (Mexomere | 0.75 | 0.25 | 0.25 | 0.25 | 0.25 |
| PP from CHIMEX) | | | | | |
| Styrene/ethylene/butylene | — | 0.5 | — | 0.5 | 0.5 |
| triblock copolymer (Kraton G- | | | | | |
| 1650E from SHELL) | | | | | |
| Dispersion of poly(methyl | 50 | 51.5 | 51.5 | 51.5 | 25 |
| methacrylate/acrylic acid) | | | | | |
| particles stabilized on the | | | | | |
| surface, in isododecane, with a | | | | | |
| polystyrene/copoly(ethylene- | | | | | |
| propylene) sequence diblock | | | | | |
| copolymer, sold under the | | | | | |
| name KRATON G1701 with a | | | | | |
| polymer solids content of | | | | | |
| 24.5% (Mexomere PAP from | | | | | |
| Chimex) | | | | | |
| Pigments (black iron oxides) | 4.6 | 3.5 | 3.5 | 3.5 | 3.5 |
| Propylene carbonate | 1.74 | — | — | — | — |
| Preservatives | qs | qs | qs | qs | qs |
| Isododecane | qsp 100 | qsp 100 | qsp 100 | qsp 100 | qsp 100 |

The above stated percentages are expressed as percentages by weight of the starting materials in question.

The rubbing resistance of these formulations is shown in Table II below and was evaluated according to the following protocol provided below.

The results show that only the compositions according to the disclosure exhibit a satisfactory resistance.

TABLE II

| | | Comparative | Comparative | Comparative | | |
|---|---|---|---|---|---|---|
| | | Formulation | Formulation | Formulation | | |
| | | No. 1 | No. 2 | No. 3 | F1* | F2* |
| Film-forming | Poly(methyl | 50 | 51.5 | 51.5 | 51.5 | 25 |
| polymer | methacrylate/ | | | | | |
| | acrylic acid) | | | | | |
| | stabilized on the | | | | | |
| | surface, in | | | | | |
| | isododecane, with | | | | | |
| | a polystyrene/ | | | | | |
| | copoly(ethylene- | | | | | |
| | propylene) | | | | | |
| | sequence diblock | | | | | |
| | copolymer | | | | | |
| | (Mexomere PAP | | | | | |
| | from CHIMEX) | | | | | |
| Polymer with | Styrene/ethylene/ | — | 0.5 | — | 0.5 | 0.5 |
| styrene | butylene triblock | | | | | |
| groups | copolymer | | | | | |
| | (Kraton G-1650E | | | | | |
| | from SHELL) | | | | | |
| Polymer with a | Stearate of | — | — | 0.6 | 0.6 | 0.6 |
| crystallizable | poly(12-hydroxystearic | | | | | |
| part | acid) | | | | | |
| | oligomer | | | | | |
| | (Solsperse 21000 | | | | | |
| | from AVECIA) | | | | | |

| | | | | | |
|---|---|---|---|---|---|
| Water rubbing test | 7 | 9 | 7 | 20 | 20 |

*According to the disclosure

Stability

As stated above, the stability of the compositions according to the disclosure may be significantly improved compared with that exhibited by conventional make-up compositions, for example, of the mascara type.

This stability is assessed according to different protocols. In the case of the present disclosure, the tests provided herein was to estimate the resistance of a film to rubbing with water.

For example, in the first test, standardized specimens of false eyelashes comprising Caucasian hair were made up according to the following protocol: 3 times 10 brush strokes with drying for 2 minutes between consecutive series, followed by drying for one hour at room temperature (25° C.).

The specimens were then squeezed with a cotton disk soaked in water.

Evaluation of the resistance comprised of counting the number of disks needed to break up the mascara film. The start of disintegration corresponded to the appearance of traces of mascara on the cotton. The precision of this method was estimated with an error delta of ±2 disks.

Although the present disclosure herein has been described with reference to particular embodiments, it is to be understood that these embodiments are merely illustrative of the principles and applications of the present disclosure. It is, therefore, to be understood that numerous modifications may be made to the illustrative embodiments and that other arrangements may be devised without departing from the spirit and scope of the present disclosure as defined by the appended claims.

1. A composition comprising, in a solvent medium, (i) at least one film-forming polymer, (ii) at least one polymer comprising at least one styrene unit, wherein the at least one polymer is different from the at least one film-forming polymer (i), and (iii) at least one additional polymer soluble in the solvent medium comprising at least one crystallizable part, wherein the at least one additional polymer is different from the at least one film-forming polymer (i).

2. The composition according to claim 1, wherein the at least one film-forming polymer (i) is present in the composition in a form of a dispersion of polymer particles stabilized on a surface in a liquid fatty phase.

3. The composition according to claim 1, wherein the at least one film-forming polymer (i) is chosen from free radical polymers, polycondensates, polymers of natural origin, and mixtures thereof.

4. The composition according to claim 1, wherein the at least one film-forming polymer (i) is chosen from polyurethanes, polyetherpolyurethanes, acrylic polyurethanes, polyesters, polyesteramides, polyesters with an alkyl fatty chain, acrylic and vinylic polymers and copolymers, silicone polymers, fluorinated polymers, polyureas, polyureapolyurethanes, polyesterpolyurethanes, polyacrylamides, and mixtures thereof.

5. The composition according to claim 2, wherein the dispersion of polymer particles are stabilized with a stabilizer chosen from sequence polymers, graft polymers, random polymers, and mixtures thereof.

6. The composition according to claim 5, wherein the stabilizer is chosen from silicone polymers grafted with a hydrocarbon chain; hydrocarbon polymers grafted with a silicone chain; graft and sequence block copolymers comprising at least one block of a polyorganosiloxane type and at least one block of a free radical polymer; graft and sequence block copolymers comprising at least one block of a polyorganosiloxane type and at least one polyether; copolymers of C₁-C₄-alkyl (meth)acrylates and C₈-C₃₀-alkyl (meth)acrylates; graft and sequence block copolymers comprising at least one block resulting from the polymerization of ethylenic monomers having at least one optionally conjugated double bond, and at least one block of a styrene polymer; graft and sequence block copolymers comprising at least one block resulting from the polymerization of ethylenic monomers, and at least one block of an acrylic polymer; and graft and sequence block copolymers comprising at least one block resulting from the polymerization of an ethylenic monomer and at least one block of a polyether.

7. The composition according to claim 5, wherein the stabilizer is identical to the at least one polymer comprising at least one styrene unit (ii).

8. The composition according to claim 1, wherein the at least one film-forming polymer (i) is present in a solids content ranging from 0.1% to 55% by weight, based on the total weight of the composition.

9. The composition according to claim 2, wherein the liquid fatty phase is chosen from hydrocarbon; fluorinated; and silicone oils wherein the liquid fatty phase is chosen from mineral, animal, vegetable and synthetic origin; and mixtures thereof.

10. The composition according to claim 2, wherein the liquid fatty phase is chosen from
non-aqueous liquid compounds having a global solubility parameter according to HANSEN solubility space that is less than 17 (MPa)^{1/2},
monoalcohols having a global solubility parameter according to HANSEN solubility space that is less than or equal to 20 (MPa)^{1/2}, and
mixtures thereof.

11. The composition according to claim 1, wherein the at least one polymer comprising at least one styrene unit (ii) is a copolymer comprising at least one block resulting from the polymerization of at least one ethylenic monomer having at least one optionally conjugated double bond, and at least one block of a styrene polymer.

12. The composition according to claim 11, wherein the copolymer is chosen from “diblock” and “triblock” copolymers of the polystyrene/polyisoprene, polystyrene/polybutadiene, and polystyrene/copoly(ethylene-propylene) types.

13. The composition according to claim 1, wherein the at least one crystallizable part of the at least additional one polymer that is soluble in the solvent medium (iii) is present in an amount of at least 5% by weight, based on the total weight of the additional polymer that is soluble in the solvent medium (iii).

14. The composition according to claim 1, wherein the at least one additional polymer that is soluble in the solvent medium (iii) is chosen from copolymers of saturated linear C₁₂ to C₃₀ alkyl (meth)acrylates and linear C₄ to C₁₀; branched, cyclic and unsaturated C₄ to C₃₀ alkyl (meth)acrylates; copolymers of vinyl esters with saturated linear C₁₂ to C₃₀ alkyl groups and vinyl esters with linear C₄ to C₁₀; branched, cyclic and unsaturated C₄ to C₃₀ alkyl groups; polycondensates of a polyamide type resulting from condensation between (α) at least one acid chosen from dicarboxylic acids having at least 32 carbon atoms and (β) an alkylenediamine, the polycondensate comprising at least one terminal carboxylic acid group esterified and amidified with at least one monoalcohol and monoamine having from 12 to 30 saturated linear carbon atoms; and lipophilic polyester polycondensates whose ends are esterified with a crystallizable acid and alcohol comprising a saturated linear C₁₂ to C₃₀ carbon chain.

15. The composition according to claim 1, wherein the at least one additional polymer that is soluble in the solvent medium (iii) is chosen from vinyl acetate/vinyl stearates, vinyl acetate/allyl stearates, vinyl acetate/ethylene and ethylene/maleic anhydride copolymers, hydrogenated butadiene/isoprene sequence copolymers, and poly(12-hydroxystearic acid) with at least one end is esterified with stearic acid.

16. The composition according to claim 1, wherein the at least one polymer comprising at least one styrene unit (ii) is present in an amount ranging from 0.01% to 30%, based on the total weight of the composition.

17. The composition according to claim 1, wherein the at least one additional polymer that is soluble in the solvent medium (iii) is present in an amount ranging from 0.01% to 30% by weight, based on the total weight of the composition.

18. The composition according to claim 1, wherein the composition comprises:
from 20% to 50% by weight of the at least one film-forming polymer (i), from 0.1% to 10% by weight of the at least one polymer comprising at least one styrene unit (ii), and
from 0.1% to 10% by weight of the at least one additional polymer that is soluble in the solvent medium (iii).

19. The composition according to claim 1, wherein the solvent medium is a non-aqueous medium comprising at least one volatile, water-insoluble compound that is liquid at room temperature.

20. The composition according to claim 19, wherein the at least one volatile, water-insoluble compound is chosen from hydrocarbon, silicone and fluorinated oils, organic solvents, and mixtures thereof.

21. The composition according to claim 19, wherein the at least one volatile, water-insoluble compound is present in the composition in an amount ranging from 5% to 55% by weight, based on the total weight of the composition.

22. The composition according to claim 1, wherein the composition comprises a content of water and/or at least one water-soluble solvent that is greater than or equal to 0.5% by weight, based on the total weight of the composition.

23. The composition according to claim 1, further comprising at least one wax.

24. The composition according to claim 23, wherein the at least one wax is chosen from waxes that are solid and rigid at room temperature and have a melting point greater than or equal to 30° C.

25. The composition according to claim 23, wherein the at least one wax is chosen from hydrocarbon waxes and esters thereof; waxes obtained by the catalytic hydrogenation of animal and vegetable oils having linear and branched C₈-C₃₂ fatty chains; and waxes obtained by the hydrogenation of castor oil esterified with cetyl alcohol.

26. The composition according to claim 23, wherein the at least one wax is chosen from C₂₀-C₄₀-alkyl hydroxystearyloxystearates.

27. The composition according to claim 23, wherein the composition comprises a total wax content ranging from 10% to 70% by weight, based on the total weight of the composition.

28. The composition according to claim 1, further comprising at least one colorant.

29. The composition according to claim 1, further comprising at least one filler.

30. The composition according to claim 1, further comprising at least one cosmetically acceptable additive chosen from antioxidants, preservatives, perfumes, neutralizers, plasticizers, fibers, gelling agents, cosmetic active ingredients, and mixtures thereof.

31. A method of making up keratin fibers comprising applying a composition comprising, in a solvent medium, (i) at least one film-forming polymer, (ii) at least one polymer comprising at least one styrene unit, wherein the at least one polymer is different from the at least one film-forming polymer (i), and (iii) at least one additional polymer soluble in the solvent medium comprising at least one crystallizable part, wherein the at least one additional polymer is different from the at least one film-forming polymer (i) to the keratin fibers.
